(19) United States
(12) Patent Application Publication
(10) Pub. No.: US 2026/0226528 A1
(43) Pub. Date: Aug. 6, 2026

(54) HYBRIDISATION DETECTION

(71) Applicant: EVONETIX LTD, Cambridge, Cambridgeshire (GB)

(72) Inventors: Matthew James HAYES, Cambridge, Cambridgeshire (GB); Vijay NARAYAN, Cambridge, Cambridgeshire (GB); Raquel SANCHES-KUIPER, Cambridge, Cambridgeshire (GB)

(21) Appl. No.: 19/148,163

(22) PCT Filed: Jan. 12, 2024
(86) PCT No.: PCT/GB2024/050086

§ 371 Date: Jul. 15, 2025

(30) Foreign Application Priority Data
Jan. 16, 2023 (GB) 2300626.5

(51) Int. Cl. C12Q 1/6825; C12Q 1/6806; C12Q 1/6841

(52) U.S. Cl. CPC C12Q 1/6825; C12Q 1/6806; C12Q 1/6841

(57) ABSTRACT

A method is provided for detecting a hybridisation indication indicative of an amount of hybridisation between a set of probe biomolecules provided at a reaction site and a set of sample biomolecules. The method includes supplying the set of sample biomolecules to the reaction site comprising the set of probe biomolecules. The reaction site is heated to cause dissociation of probe/sample-biomolecule-pairs previously hybridized when the set of sample biomolecules were provided to the reaction site. The hybridisation indication is generated based on detecting the dissociation of the probe/sample-biomolecule pairs caused by heating the reaction site.

The present technique relates to the field of biomolecules.

A sample biomolecule may undergo a hybridisation reaction with a probe biomolecule when the sample biomolecule and probe biomolecule are complementary to each other, forming a hybridised probe/sample biomolecule pair. Detection of whether hybridisation occurs between an unknown sample biomolecule and a known probe biomolecule may be used to provide an indication of whether the sample biomolecule matches a desired target biomolecule which is complementary to the probe biomolecule. However, hybridisation may be difficult to detect, requiring the use of complex detection equipment. It would be desirable to provide an improved hybridisation detection method.

Viewed from one aspect, the present technique provides a method for detecting a hybridisation indication indicative of an amount of hybridisation between a set of probe biomolecules provided at a reaction site and a set of sample biomolecules, the method comprising:
supplying the set of sample biomolecules to the reaction site comprising the set of probe biomolecules;
heating the reaction site to cause dissociation of probe/sample-biomolecule-pairs previously hybridized when the set of sample biomolecules were provided to the reaction site; and
generating the hybridisation indication based on detecting the dissociation of the probe/sample-biomolecule pairs caused by heating the reaction site.

Viewed from a further aspect, the present technique provides use of a thermal control chip, comprising a plurality of reaction sites and temperature control circuitry to independently control temperature for respective reaction sites, for increasing signal strength in detection of hybridisation between a set of sample biomolecules and respective sets of probe biomolecules provided at the respective reaction sites.

Viewed from a further aspect, the present technique provides a method for detecting hybridisation between a set of sample biomolecules and respective sets of probe biomolecules provided at respective reaction sites of an apparatus comprising temperature control circuitry to independently control temperature for respective reaction sites; the method comprising:
for each reaction site alternately selected as a selected reaction site, alternately performing a detection process comprising:
   controlling the temperature at the respective reaction sites to set the selected reaction site to a different temperature to other reaction sites;
   detecting a detection signal indicative of an amount of hybridisation between the set of probe biomolecules and the sample biomolecules, wherein the detection signal is incapable of resolving which particular site is the selected reaction site at which the hybridisation or dissociation occurred; and
   based on the detection signal, recording a hybridisation indication associated with the selected reaction site.

Viewed from a further aspect, the present technique provides use of a thermal control chip, comprising a plurality of reaction sites and temperature control circuitry to independently control temperature for respective reaction sites, for multiplexing a plurality of reaction sites to detect hybridisation between a set of sample biomolecules and a set of probe biomolecules based on a detection signal indicative of an amount of hybridisation across the thermal control chip as a whole.

Further aspects, features and advantages of the present technique will be apparent from the following description of examples, which is to be read in conjunction with the accompanying drawings, in which:

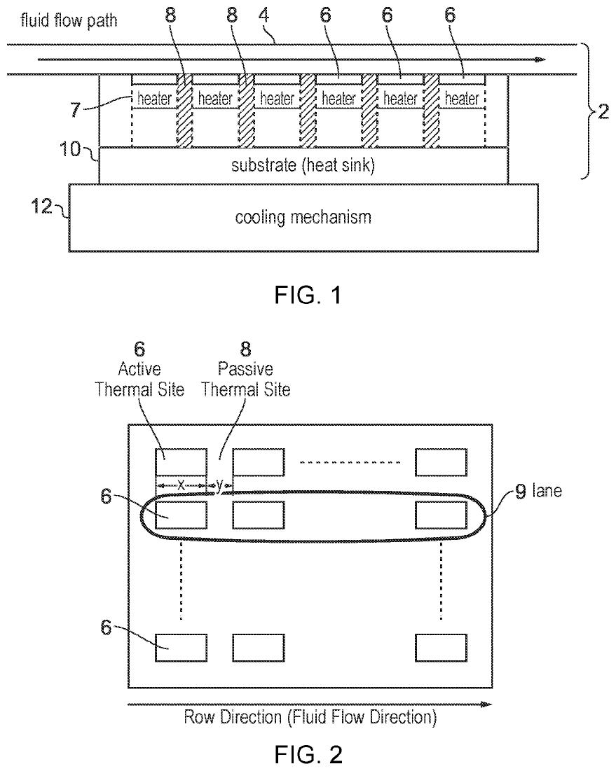

FIG. 1 illustrates a section view of an apparatus which may be used in the detection of hybridisation of biomolecules.

FIG. 2 illustrates a plan view of the apparatus shown in FIG. 1.

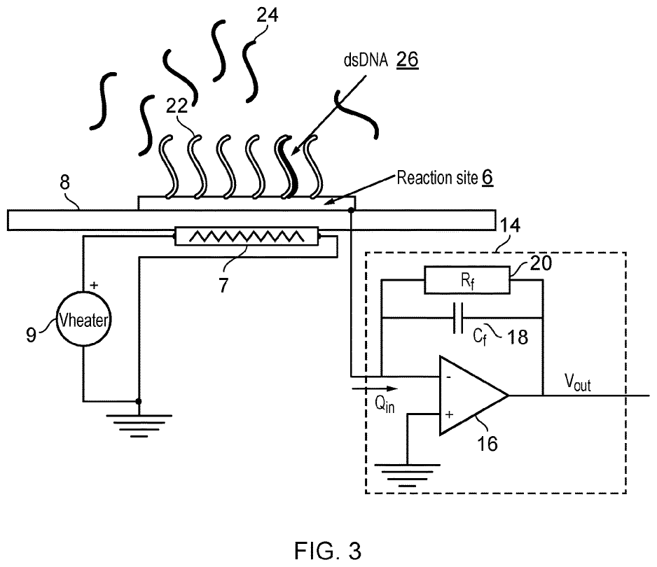

FIG. 3 schematically illustrates a reaction site at which dissociation of hybridised biomolecules may be detected.

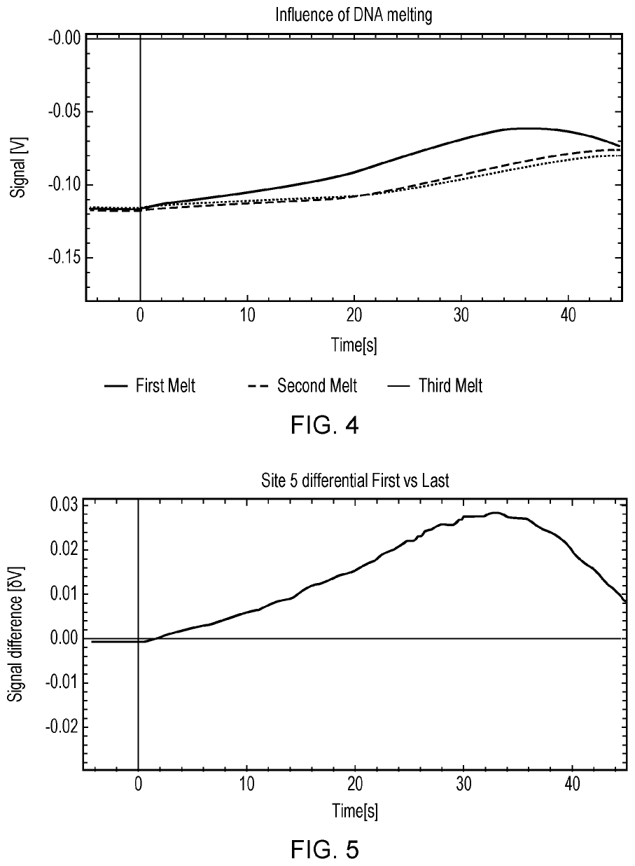

FIG. 4 is a graph illustrating a detection signal of electrical detection circuitry during heating of a reaction site.

FIG. 5 is a graph illustrating the difference between a signal associated with a first heating process and a signal associated with a further heating process at a reaction site.

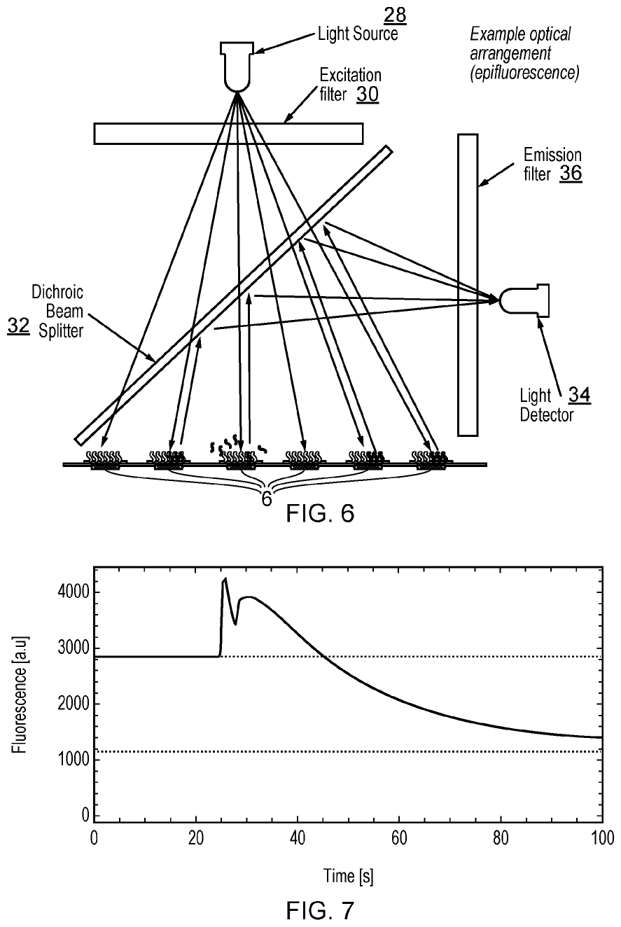

FIG. 6 illustrates fluorescence detection circuitry for detecting fluorescence at a plurality of reaction sites.

FIG. 7 is a graph showing an amount of fluorescence measured at a site containing bound probe/sample biomolecule pairs during a first heating process.

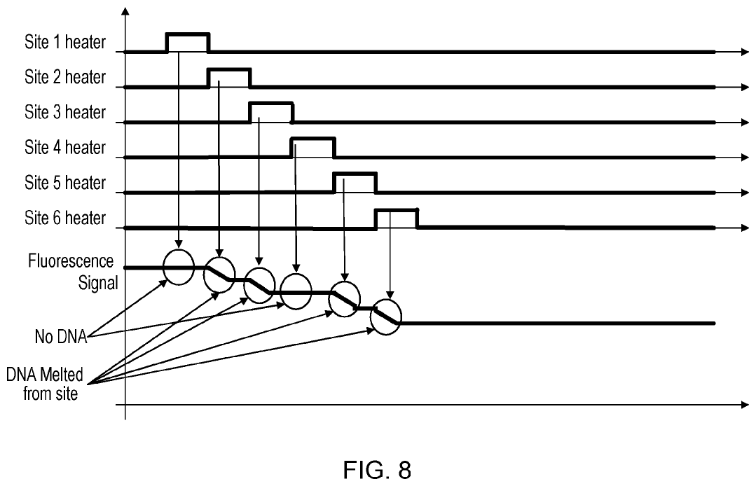

FIG. 8 shows a series of graphs illustrating the use of fluorescence detection for determining whether each of six reaction sites contain hybridised biomolecules using detection circuitry unable to distinguish between the reaction sites.

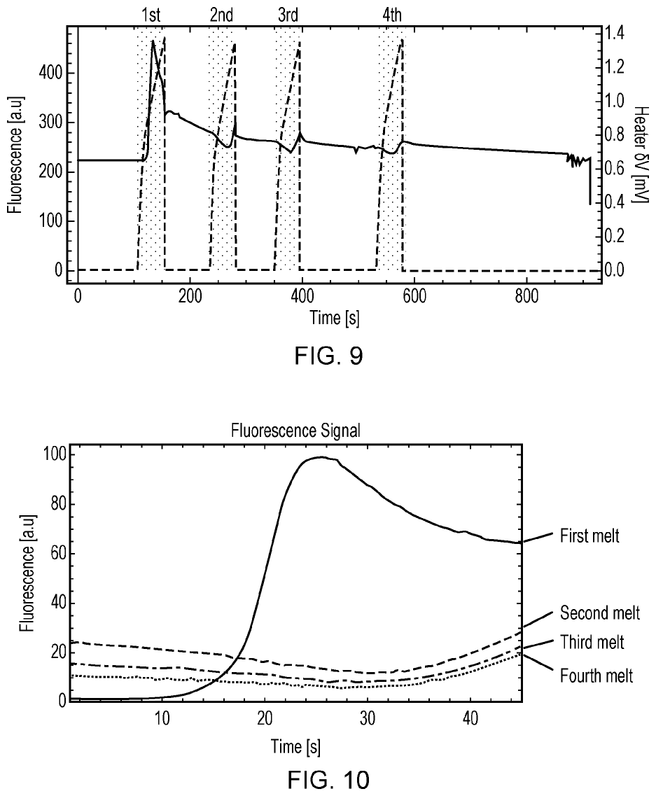

FIG. 9 is a graph illustrating the change in fluorescence at a particular reaction site initially containing bound probe/sample biomolecule pairs during four heating processes.

FIG. 10 is a graph illustrating the change in fluorescence at a particular reaction site initially containing bound probe/sample biomolecule pairs during four heating processes, in which the fluorescence signal during the heating processes has been isolated.

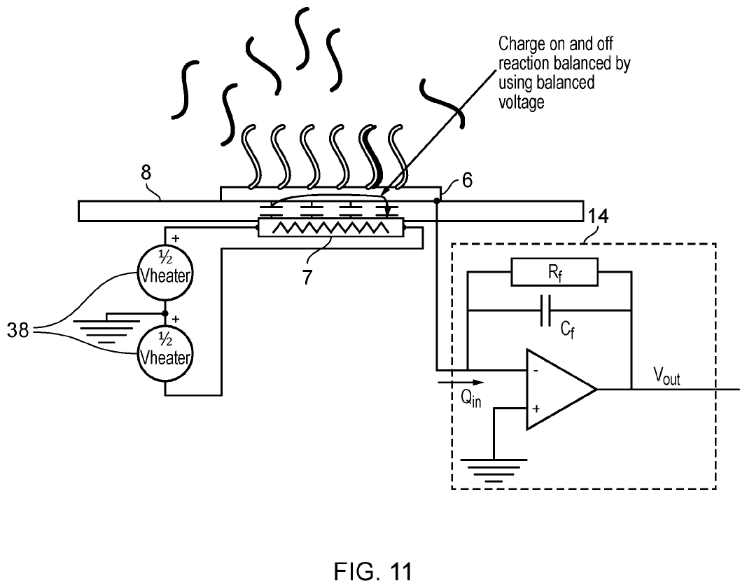

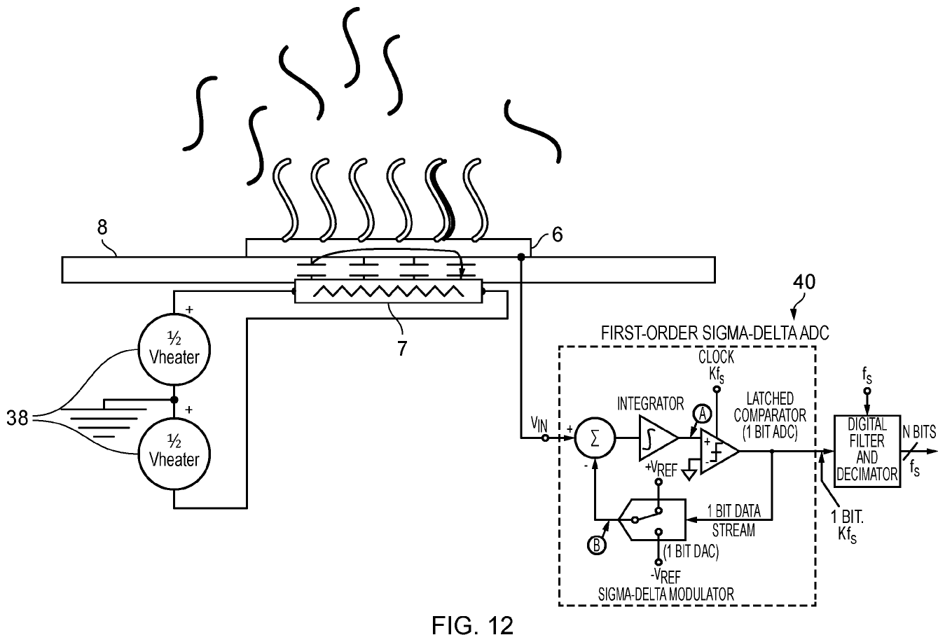

FIGS. 11 and 12 are schematic diagrams illustrating further examples of a reaction site at which dissociation of hybridised biomolecules may be detected.

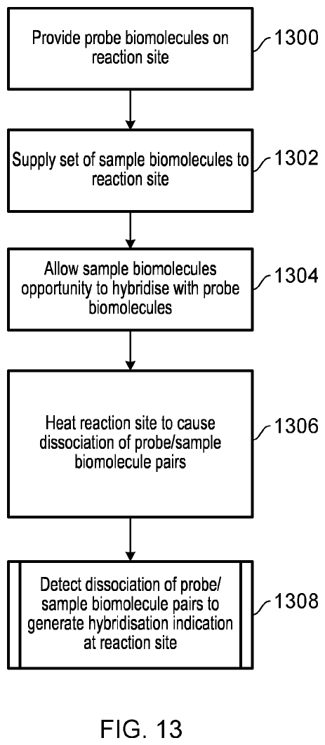

FIG. 13 is a flow diagram illustrating a method for detecting hybridisation by measuring dissociation of hybridised probe/sample biomolecule pairs.

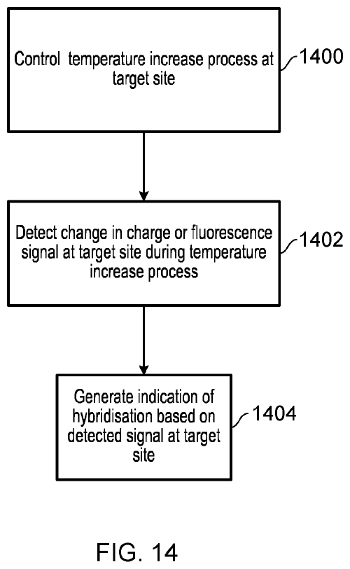

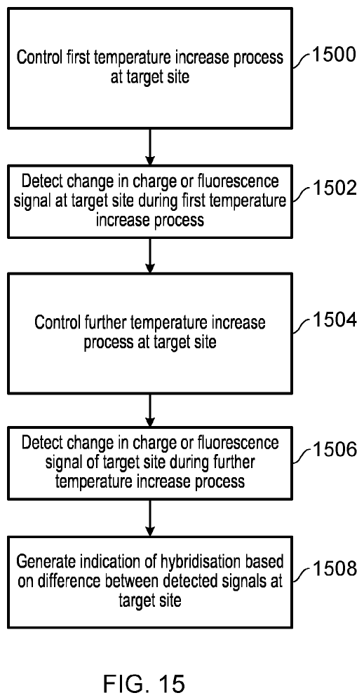

FIGS. 14 and 15 are flow diagrams illustrating in further detail the processed of heating a reaction site and generating a hybridisation indication based on a detected signal.

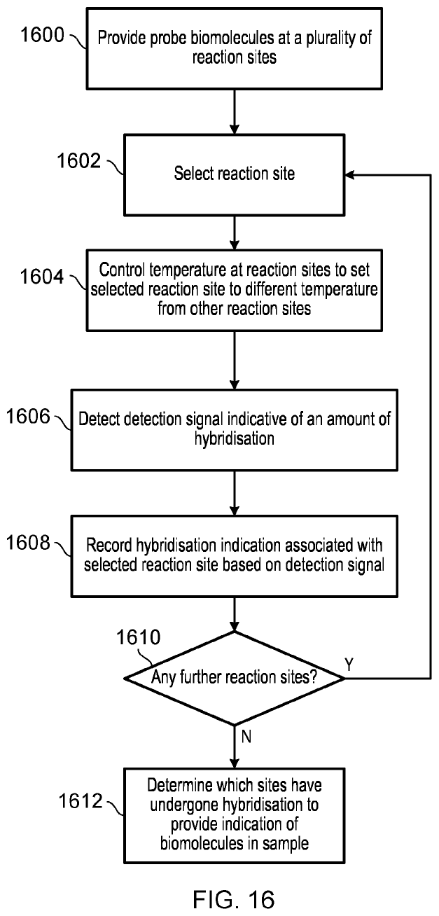

FIG. 16 is a flow diagram illustrating a method for generating a hybridisation indication for each of a plurality of reaction sites using detection circuitry which is unable to distinguish between reaction sites.

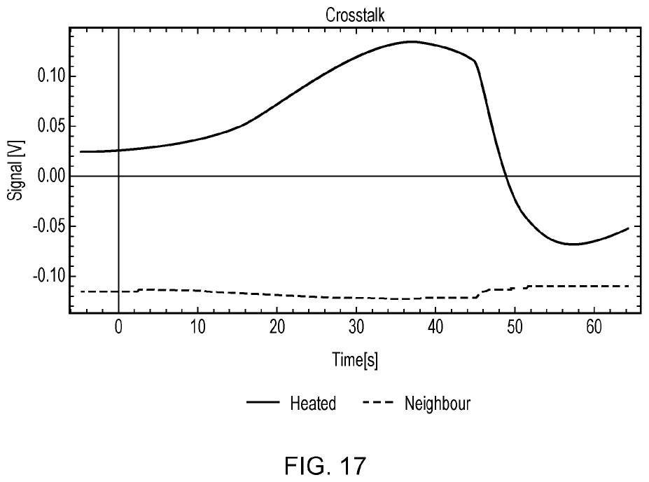

FIG. 17 is a graph illustrating output signals of electrical detection circuitry of two nearby sites in an example thermal control chip comprising a plurality of reaction sites.

A method is provided for detecting an amount of hybridisation of a set of sample biomolecules with a set of probe biomolecules. For example, a given probe biomolecule may be complementary to a given target biomolecule of interest. Hence, if it can be determined how much an unknown set of sample biomolecules hybridises with a given probe biomolecule whose identity is known, then this provides information for identifying the set of sample biomolecules. In one example detection process, a set of sample biomolecules could be exposed to a range of different probe biomolecules and determining an amount of hybridisation between the set of sample biomolecules and each of the probe biomolecules can provide an indication of whether the set of sample biomolecules includes the target biomolecules corresponding to the probe biomolecules.

When separate probe and sample biomolecules become bound (hybridise) to form a probe/sample biomolecule pair, there may be changes in measurable physical properties in the vicinity of the bound pair. For example, if a sample biomolecule has an electric charge, then when the sample biomolecule becomes bound to a probe biomolecule, there may be a localised change in the amount of charge. Similarly, if a sample biomolecule that is fluorescent (or is bound with a fluorescent substance) becomes bound to a probe biomolecule, then the probe/sample biomolecule pair may be fluorescent when the probe biomolecule was not previously fluorescent.

One method for detecting hybridisation may therefore be to measure changes in physical properties when a set of sample biomolecules is introduced to, and allowed to undergo hybridisation with, a set of probe biomolecules. For example, if smaller changes in physical properties are detected then it may be determined that a smaller amount of hybridisation has occurred. This may indicate that the sample does not include biomolecules complementary to the particular set of probe biomolecules, and further tests can be repeated with different sets of probe biomolecules. When a physical change is detected indicating that a larger amount of hybridisation has occurred, then it may be inferred that the sample biomolecule includes a biomolecule complementary to the selected probe biomolecule, allowing the sample biomolecule to be identified (or at least allow the identity to be narrowed down to a set of biomolecules complementary with the particular probe biomolecule).

However, the rate of hybridisation between a set of sample biomolecules and a set of probe biomolecules is typically slow. Over the period of hybridisation, the total number of probe/sample biomolecule pairs that have been formed may slowly increase until an equilibrium has been reached. As the rate of hybridisation is slow, the rate of change in physical properties associated with hybridisation is expected to be low. Measurable quantities may be associated with a rate of change of a physical property such that low rates of change of physical properties may cause small measurable values. For example, a measurable current may be related to a rate of change of charge. Since the signals associated with hybridisation are typically small, the signals may also have a low signal-to-noise ratio (SNR). Hence, it may be practically difficult to directly measure the changes associated with the process of hybridisation. In addition to the practical difficulties, the circuitry required to make measurements which can identify hybridisation may also be complex and expensive. For example, electrical measurements for identifying hybridisation may rely on ISFETs (ion sensitive field effect transistors) which can be expensive and complex to manufacture. Furthermore, the presence of screening signals (such as screening charges) associated with the sample biomolecules during hybridisation can obscure the hybridisation signals, further increasing the challenge of measuring hybridisation. Hence, it is desirable to provide an alternative method for detecting hybridisation.

When the temperature of a bound probe/sample biomolecule pair is increased, it becomes more likely that the pair will dissociate back into separate probe and sample biomolecules. For example, there may be a “melt” temperature above which it is much more likely that the pair will dissociate. When dissociating, the changes in physical properties that occurred on binding may be reversed. For example, charge may be released or there may be changes in the amount of fluorescence at the probe biomolecules. The inventors have realised that the changes occurring on dissociation may occur more quickly than the changes during hybridisation. In particular, whilst the rate of hybridisation is low, the rate of dissociation may be high as a large proportion of hybridised pairs begin to dissociate at similar times when a temperature is reached where dissociation becomes much more likely. The timescale for dissociation of a set of biomolecule pairs may be much shorter than the timescale for hybridisation of a set of biomolecules. The inventors have realised that measurable quantities can be orders of magnitude larger during dissociation than during hybridisation, and have realised that this offers an opportunity to detect hybridisation with reduced practical difficulties.

In one example of the present technique, a set of sample biomolecules is supplied to a reaction site comprising a set of known probe biomolecules. The conditions at the reaction site may favour hybridisation such that hybridisation is expected to occur if the set of sample biomolecules are complementary to the set of probe biomolecules.

After hybridisation has had an opportunity to take place, the reaction site may be heated to cause dissociation of probe/sample biomolecule pairs that have formed at the site since the set of sample biomolecules were provided to the reaction site. A hybridisation indication is detected based on detecting the dissociation of probe/sample biomolecule pairs caused by heating the reaction site. For example, the dissociation may be detected by measuring physical quantities such as changes in electrical charge and/or fluorescence at the reaction site. By measuring an effect of the dissociation, this can provide a quantity indicative of the amount of hybridisation that occurred when the set of sample biomolecules were introduced to the reaction site. For example, if a smaller amount of hybridisation took place then there would be fewer probe/sample biomolecule pairs at the reaction site to dissociate and therefore a smaller dissociation signal, whereas if a greater amount of hybridisation took place then there would be a greater number of probe/sample pairs at the reaction site and therefore a larger amount of dissociation to measure. Hence, a hybridisation indication indicating an amount of hybridisation that took place between the set of sample biomolecules and the set of probe biomolecules can be generated on the basis of the dissociation measurement.

As discussed above, since there is a larger rate of change in the number of hybridised probe/sample biomolecule pairs at the reaction site during dissociation than during hybridisation, a measurement of an effect of dissociation is easier to perform in practice than measuring hybridisation directly. The signals associated with dissociation may be larger, and have a higher signal-to-noise ratio. As such, simpler and lower cost measurement circuitry may be employed. Also, there may be reduced screening signals (such as screening charges) during dissociation as the unbound sample biomolecules may have already been removed after hybridisation, further reducing the measurement noise. Therefore, indirect hybridisation detection by detecting subsequent dissociation caused by heating provides an improved method for detecting hybridisation.

In some examples, the hybridisation indication may be a continuous value providing a quantitative indication of an amount of hybridisation that has occurred at the reaction site. However, in some other examples a threshold could be applied to distinguish measured values indicating little or no hybridisation occurring at the reaction site and measured values indicating that greater than a threshold amount of hybridisation has occurred at the reaction site. Hence, the hybridisation indication may be a binary indication of whether a given amount of hybridisation has occurred at the reaction site.

Which physical property is measured to provide an indication of dissociation is not particularly limited. Different approaches may exploit different physical effects of dissociation.

As identified above, in some examples the dissociation of the probe/sample biomolecule pairs is detected electrically, based on charge released during dissociation. The measurement circuitry may detect change in the amount of charge at the reaction site, for example. Whilst the detection circuitry for detecting the electrical charge may be located at the reaction site to directly measure the reaction site, this is not necessary and instead a path may be provided from the reaction site to the detection circuitry. The path may be an electrically conductive path such as a wire to carry electrical signals to the detection circuitry.

In some implementations, charge may be deposited at the reaction site as a side-effect of providing current to heating circuitry for heating the site. In some instances, the rate of charge deposited by the heating circuitry may be larger than, and obscure, the rate of change of charge at the reaction site caused by dissociation of probe/sample biomolecule pairs. Hence, in some examples it may be useful to account for the change in charge associated with the heating circuitry.

Therefore, in some examples heating the reaction site comprises suppressing the rate of change of charge at the reaction site caused by the heating circuitry. A number of different techniques may be available for suppressing the rate of change of charge caused to be introduced to the reaction site by the heating circuitry. For example, with an implementation based on resistive heating due to current passing through a path with a certain amount of electrical resistance, the rate of change in voltage across the heating circuitry may be suppressed when changing temperature, which will cause less current to flow through the heating circuitry and hence reduce the rate of leakage of charge from the heater to the reaction site. An alternative example (which has the benefit of supporting faster changes of temperature) is to suppress the rate of change of charge caused by the heating circuitry by using parallel heater circuits controlled by symmetrical voltage sources with equal and opposite voltages, such that the net rate of change of charge at the reaction site is reduced because currents driven by opposite voltages at least partially cancel each other out in terms of net charge deposited onto or removed from the reaction site, but provide a combined heating effect proportional to the total amount of current regardless of current direction. Either way, by suppressing the rate of change of charge at the reaction site caused by the temperature control circuitry, the rate of change of charge caused by the dissociation may be easier to detect, and require less precise detection circuitry.

In some other examples, detection of the dissociation of probe/sample biomolecule pairs may be made based on a measured change in fluorescence. For example, the reaction site may be illuminated with light of a particular excitation wavelength and an amount of light at a fluorescent wavelength may be measured. The change in fluorescence may be measured in the probe biomolecules, the sample biomolecules, or an intercalating dye. For example, the sample biomolecules or an intercalating dye bound to the sample molecules may be fluorescent. When bound probe/sample pairs at the reaction site dissociate, the level of fluorescence at the reaction site may increase due to the reduced quenching of fluorescence as the unbound fluorescent sample biomolecules are transported away from the reaction site. However, this is a transient effect, and after the unbound biomolecules have been transported away from the reaction site (e.g., under the influence of an electric field or by transport in a fluid) the amount of fluorescence remaining at the reaction site may be lower than before dissociation due to a reduction in an amount of fluorescent biomolecules at the reaction site (if an amount of fluorescent sample biomolecules or dye at the reaction site has been reduced by the dissociation). Hence, either or both of a temporary increase and a longer term decrease in fluorescence may be measured to indicate that dissociation has occurred. In either case, the rate of change of fluorescence caused by the dissociation is expected to be larger than the rate of change during hybridisation, and hence measuring fluorescence on heating the reaction site provides a method for easier detection of hybridisation requiring lower accuracy than directly measuring hybridisation.

It will be appreciated that whilst electrical charge and fluorescence have been described as two examples of physical properties which may be measured during dissociation to identify hybridisation, these are merely intended to be examples and the principle that rate of change is larger, and therefore easier to measure, during dissociation than during hybridisation also applies to other physical properties.

Whilst the description up to this point has only considered a single reaction site, in practice it may be beneficial to detect dissociation at a plurality of reaction sites in the same process. This is because it may be desirable to expose the set of sample biomolecules to a number of different sets of probe biomolecules to identify the sample biomolecules. Rather than repeating the entire process multiple times using a single probe biomolecule each time until a match is detected or until the different sets of probe biomolecules have been exhausted, it can be more efficient to expose the set of sample biomolecules to a number of reaction sites containing different sets of probe biomolecules at the same time, and then determine whether each of the sets of probe biomolecules has hybridised with the set of sample biomolecules. Hence, the reaction site described above may in some examples be one of a plurality of reaction sites of an apparatus comprising temperature control circuitry to control temperature for respective reaction sites, where respective reaction sites comprise respective sets of probe biomolecules (e.g., different sets of probe biomolecules). The set of sample biomolecules may be provided to each of the plurality of reaction sites and provided with the opportunity to hybridise with each of the reaction sites, for example simultaneously. For example, a fluid comprising the set of sample biomolecules may be provided to a surface or vessel comprising the plurality of reaction sites.

When there are a plurality of reaction sites comprising different sets of probe biomolecules, it may be desirable to measure a hybridisation detection signal for each reaction site. If all sites were heated at the same time then dissociation of pairs at several sites may happen simultaneously. In one example, detection circuitry may have spatial resolution at least accurate enough to distinguish a signal from one reaction site from the signal associated with a different reaction site. In this example, hybridisation or dissociation could occur simultaneously and measurements could be associated with each reaction site. However, in practice the components required to provide the required level of spatial resolution may be complex and expensive. For example, in the electrical example an amplifier may need to be associated with (e.g., provided at) each reaction site to provide a measurement output, and in the fluorescent example different optical paths and optical detectors may need to be provided for each site. In both cases, measures may be required to prevent signals from one site from affecting the signals at another site.

Recognising these difficulties, the inventors have proposed a method using an apparatus having a plurality of reaction sites with temperature control circuitry to independently control temperature at each reaction site. The detection process may comprise alternately performing heating and hybridisation indication generation steps separately for each site. By heating each site alternately, the requirement for the measurement circuitry to have spatial resolution is lifted because the signals associated with each site become temporally separated. By heating each site alternately, whenever a signal is detected it can be associated with the site being heated, so that an association can be provided between reaction sites and measurement signals without requiring detection circuitry having spatial resolution. This can significantly reduce the amount of measurement circuitry required and its complexity, reducing the cost and difficulty of performing the detection method with a plurality of reaction sites.

In some examples, the method described above may be performed using reaction sites provided on a thermal control chip. The chip may have reaction sites which can be individually heated. For example, reaction sites may be provided with individually controllable heating circuitry. This means that sites can be individually addressed with the heating circuitry, enabling the use of shared dissociation detection circuitry.

In some examples, the thermal control chip may provide a plurality of reaction sites without having permanent fluidic barriers between neighbouring sites, to allow a continuous fluid comprising the set of sample biomolecules to be exposed to the plurality of reaction sites (rather than, for example, individually exposing the set of sample biomolecules to each site in turn) making it easier to provide the set of sample biomolecules to each of a plurality of reaction sites.

As discussed above, in some examples the hybridisation indication for a given reaction site is generated, using detection circuitry, based on a detection signal indicative of an amount of dissociation when the given reaction site is heated.

The detection circuitry may be provided individually for each site. In this case, performing the detection process by alternately performing the heating and generating steps for each reaction site may improve accuracy by reducing the amount of noise generated by dissociation signals at neighbouring sites. However, as discussed above, providing the detection circuitry at each site may be complex and require a large amount of detection circuitry.

Therefore, the detection circuitry may be shared between the plurality of reaction sites. For example, in examples where the detection circuitry detects an electrical signal, the reaction sites may each be electrically connected to shared electrical detection circuitry. When the detection circuitry detects a fluorescent signal, the light detection circuitry may lack the resolution required to distinguish neighbouring sites. For example, light from different sites may be combined (e.g., using a lens) and provided to a shared light detector. This simplifies and reduces the amount of detection circuitry, reducing cost, whilst maintaining the ability to associate each set of probe biomolecules with a detection signal using addressable heating circuitry.

In some examples, the detection circuitry is provided within the boundary of the thermal control chip. For example, an amplifier to convert a charge measurement into an output voltage may be provided among the reaction sites, such as at each site or connected to each site within the chip. Alternately, the detection circuitry may be provided outside the boundary of the thermal control chip. For example, the thermal control chip may comprise one or more output pins carrying an electrical detection signal from one or more reaction sites to external detection circuitry. Similarly, external light detection circuitry may detect fluorescence at the reaction sites of the chip.

In some examples, the detection signal is incapable of resolving which of the plurality of reaction sites is the given reaction site at which the probe/sample biomolecule pairs dissociated. As described above, this reduces the complexity of detection circuitry and the ability to perform hybridisation detection separately for a plurality of reaction sites can be maintained by alternately performing the heating and generation steps for each reaction site.

In some examples, the hybridisation indication for the given reaction site is generated based on the detection signal and information indicative of which reaction site was heated when the detection signal was detected by the detection circuitry.

As described above, sets of probe molecules are provided at each reaction site. The probe biomolecules may be synthesised separately as desired and then deposited onto each reaction site in advance of the hybridisation detection. The probe biomolecules may, for example, be bound to each reaction site such that the probe biomolecules or resulting probe/sample biomolecule pairs are prevented from leaving the reaction site. This forms an association between sets of probe biomolecules and physical sites, such that detection associated with a given type of probe biomolecule can be performed by addressing individual sites (e.g., by heating a given site).

In some examples, the probe biomolecules may be synthesised in situ on the reaction site prior to supplying the set of sample biomolecules. For example, the reaction sites may also be useable for the synthesis of biomolecules. The hybridisation detection process may be simplified by synthesising probe biomolecules in situ, and it may also be easier to ensure that a given set of probe biomolecules is associated with a given site, compared to attaching biomolecules to particular sites which may be more likely to lead to biomolecules becoming attached to a neighbouring site.

In some examples, the reaction site is one of a plurality of reaction sites of an apparatus comprising temperature control circuitry to independently control temperature for respective reaction sites, and synthesis of a respective set of probe biomolecules at respective reaction sites is thermally controlled by the temperature control circuitry independently controlling temperatures at each reaction site. Hence, the same temperature control circuitry may be reused to both synthesise a set of probe biomolecules and detect hybridisation between those biomolecules and a set of sample biomolecules.

In some examples, the set of probe biomolecules could comprise a nucleic acid (or a synthesis initiator of a nucleic acid or peptide conjugate/hybrid with a nucleic acid), such as deoxyribonucleic acid (DNA). There has been significant interest in creating DNA detectors for use in sequencing, gene expression analysis and other applications, and it would be desirable to provide an effective method for detecting DNA (or another nucleic acid) based on detecting hybridisation of a set of sample biomolecules with a set of probe biomolecules. Hence, in some examples, the probe biomolecules comprise at least one of:
single-stranded DNA, RNA or another nucleic acid;
synthesis initiators for DNA, RNA or another nucleic acid; and
peptides, DNA peptide conjugates, or peptide hybrids with another nucleic acid.
   In some examples, the sample biomolecules comprise at least one of:
single-stranded DNA, RNA or another nucleic acid; and
peptides, DNA peptide conjugates, or peptide hybrids with another nucleic acid.
   The sequences of said probe biomolecules may be selected arbitrarily such that they are complementary to, and synthesise with, certain desired sets of sample biomolecules allowing identification of sample biomolecules by determining which probe biomolecules undergo hybridisation. In some examples, both the set of probe biomolecules and the set of sample biomolecules comprise at least one of double-stranded or single-stranded DNA, RNA or another nucleic acid. In examples using a plurality of reaction sites, the sets of probe biomolecules bound to different sites may comprise different sequences of nucleotides, such that an amount of hybridisation at a particular site may be indicative of the presence of a particular sequence of nucleotides in the sample.

In some examples, heating the reaction site is performed once and the detection signal associated with the heating is used to generate a hybridisation indication. However, in other examples the reaction site may be heated two or more times. The hybridisation indication may then be generated based on a comparison between the first heating process and a further subsequent heating process. The inventors recognised that there may be changes in the detected signal which are not associated with dissociation of probe/sample biomolecule pairs, and which are the same in repeated measurements (for example, changes due to charge deposited at the site as a side-effect of the heating circuitry). The inventors also recognised that a majority of the bound pairs may dissociate during the first heating process. If so, the signal captured during the second or further heating process is not expected to be dominated by the dissociation signal, and is instead expected to represent the changes in detection signal associated with other factors. Hence, by calculating a difference between the two measurements, the difference is expected to represent the dissociation signal whilst removing the other factors which are the same in both measurements. Hence, noise of the dissociation signal may be reduced, and more accurate measurements may be captured with less precise measurement circuitry.

As has been discussed above, an improved method of detecting hybridisation may be provided by alternately controlling the temperature of, and detecting a detection signal at, respective reaction sites of a plurality of reaction sites. Alternately controlling the temperature of respective reaction sites allows detection circuitry to be shared between the reaction sites, without having a requirement for spatial resolution.

In some examples, the hybridisation indications generated for each of the plurality of reaction sites may indicate which of the sets of probe biomolecules at respective reaction sites have undergone hybridisation with the set of sample biomolecules. For example, a hybridisation indication for a given site indicative of a low amount of hybridisation may indicate that there has not been a large amount of hybridisation at the given site, and therefore that the probe biomolecules at that site did not undergo a large amount of hybridisation with (and therefore were not complementary with) the set of sample biomolecules.

As discussed above, in some examples the detection signal is detected based on detecting dissociation of probe/sample biomolecule pairs previously hybridised at the reaction site when the set of sample biomolecules were provided to the reaction sites. Rates of change of physical properties may be larger during dissociation than during hybridisation and therefore this may lead to more accurate and easier to obtain measurements.

Dissociation may be more likely to occur at higher temperatures, and therefore the temperature of a respective site may be controlled by heating the respective site to a higher temperature than the other reaction sites. The respective site may be set to a temperature where dissociation is much more likely to occur than at the other sites (e.g., above a “melt” temperature whilst the other sites are below the melt temperature). This means that if dissociation is measured in the plurality of reaction sites as a whole, it can be inferred that the dissociation occurred at the respective reaction site set to the higher temperature.

In some examples, instead of or in addition to measuring the detection signal based on detecting dissociation, the detection signal could be based on detecting the hybridisation itself. For example, the change in charge or change in fluorescence associated with sample biomolecules hybridising with the probe biomolecules could be measured to provide an indication of hybridisation. In particular, the measurement of hybridisation could be made by alternately controlling the temperature of a selected site to be different from that of other sites and detecting a detection signal at the selected reaction site.

To measure hybridisation directly, the selected reaction site may be set to a lower temperature than the other reaction sites. For example, the selected reaction site may be set to a temperature at which hybridisation is more likely to occur and the other reaction sites may be set to higher temperatures where hybridisation is less likely to occur. For example, the selected reaction site may be set to below a melt temperature and the other reaction sites may be set to above the melt temperature. Hence, a detected hybridisation signal can be associated with the selected site, so it can be known how much hybridisation occurs between the set of sample biomolecules and the probe biomolecules associated with the selected site even if the detection circuitry is unable to distinguish the reaction sites.

The method described above is an example of a method which may make use of a thermal control chip, comprising a plurality of reaction sites and temperature control circuitry to independently control temperature for respective reaction sites, for multiplexing a plurality of reaction sites to detect hybridisation between a set of sample biomolecules and a set of probe biomolecules based on a detection signal indicative of an amount of hybridisation across the thermal control chip as a whole. Because the association between reaction sites and detection signal is made based on independently controlling the temperature for respective reaction sites, this enables the use of detection circuitry to produce a detection signal incapable of resolving which particular site is the site at which the hybridisation occurred, and resolution of the site at which the hybridisation occurred is based on which site has its temperature set to a different temperature to other sites. Detection circuitry lacking the ability to resolve different reaction sites may be simpler and cheaper than detection circuitry having the ability to resolve different reaction sites.

Particular examples will now be described with reference to the Figures.

A technique for hybridisation detection is provided below.

FIGS. 1 and 2 illustrate an apparatus 2 (e.g. a thermal control chip) which can be used for the techniques below. As shown in FIG. 1, the apparatus has a fluid flow path 4 across the top of the device. A fluid flow element (e.g. a pump) is provided to control the flow of fluid through the fluid flow path 4. A number of reaction sites (active thermal sites) 6 are provided at various locations across the plane of the temperature control device 2. The top of each reaction site 6 may include a reaction surface (e.g. a gold cap) on which biomolecule samples (such as single-stranded or double-stranded nucleic acid fragments, fragments of other nucleic acids, synthesis initiators, or peptides) can be synthesised, attached, cleaved, or processed. Each reaction site 6 corresponds to part of a level surface, so that there is no physical barrier between adjacent reaction sites 6. Each reaction site 6 has a heating element 7 (temperature control circuitry, e.g. a resistive heater) provided below the reaction site surface to apply heat to the corresponding part of the fluid flowing over that site, to control the temperature of the fluid. The heat applied at each site is variable, based on control signals provided by an external controller. Hence, the temperature control circuitry is able to independently set the temperature for each reaction site 6. As shown in FIG. 2, the reaction sites 6 are arranged in a two-dimensional matrix (grid), e.g. arranged in two or more rows (lanes) where the lane/row direction is parallel to the direction that fluid flows through the fluid flow path 4.

The regions lying between the active thermal sites 6 form one or more passive thermal regions 8 which do not comprise any heating element, but provide passive cooling by conducting heat away from the fluid towards the substrate 10 of the device 2. The length x of each active thermal site 6 in the row direction can be longer than the length y of each passive thermal region 8 lying between a pair of adjacent active thermal sites 6 in the same row. The thermal resistance of the material provided below each active thermal site 6 in a direction perpendicular to the substrate may be greater than the thermal resistance in the direction perpendicular to the substrate of the material provided below each passive thermal region 8, which is helpful for improving the temperature range supported for a given maximum power provided by each heating element 7. As shown in FIG. 1, a cooling mechanism 12 may be provided to cool the substrate 10 to act as a heat sink. Further information about the apparatus shown in FIGS. 1 and 2, including an example of a process for manufacturing it and a control model for controlling the heating elements 7, can be found in WO 2018/104698 A1, WO 2019/064006 A1, and WO 2020/021221 A1 (the contents of which are hereby incorporated by reference).

Hence, the apparatus 2 is a thermal array allowing individual control of temperature at different regions across the two-dimensional surface of a substrate.

With such an apparatus 2, the reagents for a reaction can be supplied in the fluid travelling along the fluid flow path, to react with biomolecule samples disposed on the respective reaction sites.

The apparatus 2 may be used in a process for identifying the presence of nucleotide sequences in a sample.

Respective sets of probe biomolecules may be provided at respective sites 6 of the apparatus 2. For example each set of probe biomolecules (e.g. a fragment of DNA or RNA corresponding to a particular nucleotide sequence to be detected in a set of sample molecules) may be synthesised in situ at the site (based on the thermal control provided by the heaters 7) or may be attached to the site by other means. Further information about a method for synthesis that can be used is found in WO 2019/145713 A1 (the contents of which are hereby incorporated by reference).

To determine the presence of a particular nucleotide sequence or other property in a sample, the sample may be exposed to several sites under conditions in which the sample biomolecules may undergo hybridisation with the probe biomolecules. After exposing the sample to the probes, a process can be carried out to determine which of the sites had probe biomolecules which underwent hybridisation with the sample biomolecules, indicating which nucleotide sequences or other properties were present in the sample (e.g. as the probes favour hybridisation with complementary sequences in the sample).

However, the process for determining which probes have undergone hybridisation can be difficult, requiring relatively complex detection circuitry.

When a probe undergoes hybridisation, some electrical charge may be absorbed when bonds are formed in the hybridised product, causing a change in electrical charge localised at the probe. If each probe is associated with a physical position (for example, if they are bound at a particular location) then detection of a change in charge at that location can indicate hybridisation of the probe. However, the magnitude of the change in charge during hybridisation may be very small, and in addition the rate of change of charge can be low if the hybridisation takes place over an extended time (not all the molecules which eventually hybridise will hybridise at the same time). As electrical measurements may be dependent on the rate of change of charge which is measured, measurements can require very precise circuitry. For example, such measurements may rely on ISFETs (ion sensitive field effect transistors), which can be expensive and complex to manufacture.

Alternatively, the probes or the samples, could be tagged with a marker (such as a fluorescent marker), or an intercalating dye could be used which fluoresces when bound between probe and sample, and fluorescent microscopy imaging of the reaction apparatus may be used to identify which probes have undergone hybridisation. However, again, this process can also be difficult to carry out in practice if the marker is difficult to detect, as the hybridisation is slow and the dyes can produce a weak fluorescent signal, so resolving temporal changes in fluorescence during hybridisation and spatial differences between sites may require an extremely sensitive microscope, which increases costs.

The apparatus 2 described above may be used to more effectively determine which probes have undergone hybridisation.

In particular, each set of probe biomolecules is associated with an active thermal site 6. For example, one or more probes may be synthesised at, and bound to, the reaction surface of a corresponding active thermal site 6. The probes associated with different active thermal sites 6 may be different to determine the presence of a range of different nucleotide sequences (or other properties) in the sample. The sample may be provided to the apparatus 2 as a fluid flowing across the fluid flow path 4, exposing the sample to each of the sites (and hence each set of probes). If a given probe is complementary to a sequence in the sample, then the probe may undergo hybridisation. After the sample has been exposed to the apparatus, a number of probes (corresponding to the sequences present in the sample) may have undergone hybridisation, whilst a number of other probes not corresponding to sequences present in the sample may have not undergone hybridisation.

As discussed above, charge may become bound in the hybridised probe/sample pairs during hybridisation. When the probe/sample pair is caused to dissociate, the bound charge is released. A probe may be caused to dissociate by applying heat to the reaction sites hybridised probe. An advantage of detecting the dissociation caused by heating, rather than attempting to detect the hybridisation when the sample is previously supplied, is that when the heat is applied, many probe-sample pairs of hybridised biomolecules will dissociate in a short time, greatly increasing the signal strength of any detected electrical or optical signal compared to what could be measured during the corresponding hybridisation. As sites which underwent a greater amount of hybridisation will tend to cause a greater signal on dissociation than sites which underwent a smaller amount of hybridisation, the signal indicative of an amount of dissociation of previously hybridised pairs can be considered an approximate indication of the amount of hybridisation that previously occurred, even though it is measuring an effect of the dissociation rather than the hybridisation. Therefore, by using a detection signal detected based on the dissociation of previously hybridised probe-sample pairs, this allows the extent of hybridisation at a particular reaction site to be detected using detection circuitry with coarser resolution, being less able to finely resolve subtle changes in electrical charge/fluorescence. This reduces the cost of implementing the technique.

Therefore, one technique for determining which of the probes has been hybridised involves raising the temperature of active thermal sites 6 in the apparatus above a threshold temperature and using detection circuitry (e.g. an operational amplifier) to make an electrical measurement (e.g. of current) at the site, to determine an amount of charge released at the site after applying the heat. If the measured detection signal is less than a threshold, it may be determined that a probe corresponding to the site 6 that has been heated has not undergone hybridisation, whereas if the signal is greater than a threshold then it may be determined that the probe has undergone hybridisation to sufficient extent to judge that the sample biomolecules met the property being tested using the probe biomolecules at that site. In comparison to the process of hybridisation where charge is slowly bound to the probe, in dissociation charge may be quickly released, causing more charge to be moved per unit time. As measurement may be based on a rate of change of charge, it may therefore be easier to measure the charge that is released upon dissociation compared to the charge that is bound on hybridisation, meaning that the use of heated active sites allows for easier identification of which probes have been hybridised. Released charge may be measured distinctly for each active site or generally across the apparatus, as discussed below. A similar advantage occurs when a fluorescent imaging technique is used to capture the detection signal, as the change in brightness with time will be stronger during the heat-triggered dissociation than during the hybridisation.

Another advantage of using the thermal control apparatus 2 is for multiplexing multiple sites thermally so that less fine-grained spatial resolution is needed for the detection circuitry which measures the detection signal indicative of hybridisation between the sample/probe biomolecules. Detecting hybridisation during the hybridisation process may require charge measuring circuitry to have spatial resolution. For example, when the sample is exposed to the probes at respective sites, several probes may undergo hybridisation at the same time, and therefore circuitry may need to have the capability of detecting whether a particular probe at a given site is undergoing hybridisation, such as by measuring the change in charge in the vicinity of a particular reaction site. This requirement for spatial resolution increases the complexity of the circuitry for detecting hybridisation (e.g. requiring high-resolution microscopes or complicated ISFETs at each site).

However, identifying hybridised probes using the apparatus 2 does not require the detection circuitry to have spatial resolution, because the active thermal sites 6 are individually controllable to adjust the temperature at each site. Instead, general detection circuitry can be provided to detect a change in charge or fluorescent light across the surface of the temperature control device 2 as a whole, without the ability to pinpoint measurements to particular sites 6. The detection circuitry can be located at or beyond a boundary of the thermal control chip. The detection circuitry is shared between all reaction sites and may generate a detection signal incapable of distinguishing which particular site the signal was detected from. Hence, the electrical measurements captured at a given site can be processed using circuitry which does not distinguish which site the measurement came from (other than based on information specifying which site had the temperature at the site set differently from other sites). Alternatively, the detection of a change in fluorescent light from the chip may be performed using a single optical sensor capturing a total amount of light emitted from the chip, rather than requiring an imaging sensor with spatial resolution.

Hence, a detection process can be applied alternately to each site 6, and when the detection process is performed on a selected site, the temperature of that site is set differently to other sites, and the detection signal measured to give an indication of the hybridisation amount at that site.

In one example, the detection process involves heating the selected site to a higher temperature than other sites, to trigger dissociation of previously hybridised pairs of sample/probe biomolecules, and then the detection signal is detected to detect a change in charge or fluorescent light intensity during the dissociation.

Alternatively, during hybridisation the temperature of a particular target site may be controlled to be lower than the temperature of other sites in the apparatus 2. This may mean that hybridisation is more likely to take place for the particular cooler site (with the temperature at other sites being too high for hybridisation), and therefore if the general measurement circuitry detects a change in charge in the period when the sample biomolecules are being supplied to the chip, then it may be determined that the probe associated with the cooler site has undergone hybridisation.

Hence, by addressing individual reaction sites thermally using the individually controllable active thermal sites 6, the requirement for detection circuitry to have fine spatial resolution is lifted, and the measurement circuitry may be simplified compared to circuitry for measuring the change in charge at particular locations.

The technique described here can be useful for fields such as medical diagnostics, where a sample taken from a patient may for example be exposed to a range of probe biomolecules disposed on the reaction sites, to detect conditions associated with the sample based on which of the probe biomolecules will hybridise with the sample.

FIG. 3 illustrates a reaction site 6 (e.g., an active thermal site 6 as illustrated in FIGS. 1 and 2) comprising a heater 7 powered by a voltage source 9, and electrical detection circuitry 14. The reaction site 6 is disposed on a surface 8 of a thermal control chip, where the surface 8 may be a passive thermal region as illustrated in FIGS. 1 and 2. Although FIG. 3 illustrates the reaction site 6 extending vertically out of the surface 8, it will be appreciated that this view is merely schematic, and the reaction site may be flush with the surface 8.

The electrical detection circuitry 14 comprises an amplifier which detects a rate of change in the amount of charge at the reaction site by converting an input charge signal (Qᵢₙ) to an output voltage (Vₒᵤₜ) indicative of the rate of change in the amount of charge at the reaction site. The electrical detection circuitry 14 comprises an operational amplifier 16 connected to the reaction site 6, a capacitor 18, and a resistor 20.

The sensitivity of the electrical detection circuitry may be increased by increasing its impedance. For example, to achieve a level of sensitivity sufficient to identify changes in charge associated with dissociation of probe/sample biomolecule pairs, some examples of the electrical detection circuitry may have a relatively high impedance. In one example of the electrical detection circuitry, used to collect experimental data represented in FIGS. 4 and 5, the electrical detection circuitry had an impedance of 100 MΩ. For example, if there is a change of charge of 1 nC detected at the reaction site in a time of 10 s, then this corresponds to a current of 100 pA. With an impedance of 100 MΩ, the change in charge causes an output of 10 mV from the amplifier. The capacitor 18 in the electrical detection circuitry 14 may be selected to set the lower frequency point of the charge amplifier to a desired value. In the example electrical detection circuitry, use of a 100 nF capacitor sets the lower frequency point of the amplifier to <0.1 Hz. Increasing the gain of the output signal and applying a high-pass filter may make changes in charge easier to detect. For example, a high-pass filter with ~10 s time constant can reduce DC errors, and a gain of ~x100 can increase sensitivity. Using these values, the overall sensitivity of the example amplifier may be in the region of 1 nC/V. In some examples, the electrical detection circuitry 14 may be split into different stages. A first stage of the amplifier may be provided within the thermal control chip (e.g., at the reaction site) and a second stage of the amplifier may be provided away from the chip. For example, the second stage may further filter and amplify a signal output from the first stage.

However, it will be appreciated that this amplification circuitry is merely an example and other circuitry having the same or similar effect could be used to provide an output signal indicative of a change in electrical charge at the reaction site 6. The electrical detection circuitry 14 may be provided for each site, or may be shared between sites and selectively connected to each site using multiplexers so that it is still site-specific. In other examples the same detection circuitry 14 may be connected to reaction sites in a way which means it is incapable of distinguishing a detection signal from one site from the detection signal from another site. For example, each reaction site may be coupled to the same electrical detection circuitry 14. In such examples the heater 7 at each site 6 may be controlled to heat individual selected sites during a given detection process, so that spatial resolution of the detection circuitry 14 is not required.

FIG. 3 illustrates a set of probe biomolecules 22 bound to the surface of the reaction site 6. The set of probe biomolecules 22 may have been synthesised externally and attached to the surface of the reaction site 6, or the probe biomolecules may have been synthesised on the surface of the reaction site 6. During a hybridisation detection process, a set of sample biomolecules 24 are provided to the reaction site under conditions in which hybridisation may occur. The amount of hybridisation will be greater (typically orders of magnitude greater than if the biomolecules are not complementary to the probe molecules) if the set of sample biomolecules are complementary to the set of probe biomolecules at a given site. If the set of sample biomolecules 24 are complementary to the set of probe biomolecules 22, then there is an increased likelihood that biomolecules in the two sets of biomolecules may hybridise to form probe/sample biomolecule pairs (such as double stranded DNA) 26 bound to the reaction site. If the sets of biomolecules are not complementary, then the probability of hybridisation occurring may be significantly lower than if the sets of biomolecules were complementary, and the number of hybridised probe/sample biomolecule pairs bound to the reaction site 6 may be significantly lower. If the identity of the set of probe biomolecules is known, then information indicating whether the set of sample biomolecules contains biomolecules complementary to the set of probe biomolecules may be collected by determining an amount of hybridisation that occurs. An amount of charge at the reaction site varies during hybridisation due to charge becoming bound in the probe/sample biomolecule pairs. The charge bound in the probe/sample biomolecule pairs is released when the pairs dissociate. Dissociation may be more likely to occur at higher temperatures, especially above a threshold “melt” temperature.

During hybridisation, the rate of change of charge at the reaction site is slow due to the slow rate of hybridisation as probe biomolecules and sample biomolecules hybridise over an extended period of time. Hence, a measurable signal indicating the rate of change of charge (e.g., current) is small, and the circuitry required to detect the signal must be very accurate. However, dissociation due to heating the reaction site above the melt temperature occurs over a shorter timeframe than hybridisation as the plurality of bound pairs at the site dissociate at similar times, and therefore the rate of change of charge caused by dissociating hybridised pairs is much greater than the rate of change of charge caused by hybridising biomolecules.

Therefore, the electrical detection circuitry 14 is configured to detect a change in charge associated with dissociation, allowing it to be less precise than circuitry for directly detecting a change in charge associated with hybridisation. After the set of sample biomolecules 24 have been introduced to the reaction site 6 and allowed the opportunity to react with the set of probe biomolecules 22, the heater 7 heats the reaction site 6 above the melt temperature for the probe/sample biomolecule pairs. This causes hybridised pairs at the reaction site to dissociate and release charge. The change in charge is measured by the electrical detection circuitry 14 to provide an output voltage Vₒᵤₜ indicative of the amount of hybridisation that occurred. For example, if the output voltage has a smaller magnitude, then this may indicate that the rate of change of charge was smaller, suggesting that fewer probe/sample biomolecules were caused to dissociate and therefore that there were fewer hybridised molecules at the reaction site prior to the heating, indicating that a smaller amount of hybridisation occurred when the sample was introduced to the reaction site. This can indicate that the set of sample molecules were not complementary with the set of probe biomolecules. In comparison, if a larger magnitude output voltage is measured then this may indicate that a larger number of probe/sample biomolecule pairs were caused to dissociate, and therefore that a greater amount of hybridisation occurred when the set of sample biomolecules were previously introduced to the reaction site. Hence, a larger magnitude of output voltage can indicate that the set of sample biomolecules included biomolecules which were complementary to the set of probe biomolecules.

On a thermal control device comprising a plurality of sites (such as that shown in FIG. 2), the set of sample biomolecules may be provided to each site simultaneously. Dissociation at a given site can be measured using detection circuitry lacking the resolution to distinguish sites, by heating each site at different times. When sites are heated alternately, a single site at a time may be at a temperature where dissociation is expected to occur, and therefore dissociation signals measured anywhere in the plurality of sites may be associated with the selected site that has been heated.

FIG. 4 is a graph illustrating an output signal of the electrical detection circuitry 14 of FIG. 3 after respective heating processes. A heating process is a period of time over which the temperature of the heater 7 is increased, causing the temperature of the reaction site 6 to increase. The heating process will typically raise the reaction site from a temperature below a melt temperature to above the melt temperature.

A first heating process (“first melt”) takes place after the set of sample biomolecules have been exposed to the set of probe molecules bound to the reaction site 6. The second and third heating processes take place after the first heating process, with no additional sample biomolecules being provided to the reaction site 6 after the first melt. FIG. 4 illustrates an example in which a larger amount of hybridisation had occurred at the target reaction site, and therefore during the first melt a larger number of probe/sample biomolecule pairs were caused to dissociate and release charge. This can be observed as the output signal during the first melt increases from around −0.12V to a peak of around −0.06V.

After the first melt, there are not expected to be a significant number of probe/sample biomolecule pairs remaining at the reaction site 6, and therefore on the second and third melts there is not expected to be a significant signal caused by pairs dissociating. This can be seen by comparing the magnitudes of the second and third melts with the magnitude of the first melt.

However, the output voltage is not completely flat on the second and third heating processes. There are several potential causes of this effect which may be unrelated to the detection of hybridisation (such as charge deposited by the heater itself). One method to isolate the signal caused by dissociation from other effects is to perform a number of heating processes, and generate an indication indicative of a difference between the first heating process and a subsequent heating process. An example of this measurement is shown in FIG. 5, which shows the difference between the first and last measurements shown in FIG. 4. FIG. 5 clearly illustrates the effect of dissociation over the heating period isolated from other effects which lead to changes in charge at the reaction site during heating.

FIG. 6 illustrates circuitry which may be used to detect dissociation by measuring changes in fluorescence.

A number of reaction sites 6 are shown in FIG. 6. Each reaction site 6 may be substantially the same as the reaction site 6 shown in FIG. 3, except that the electrical detection circuitry 14 may be excluded in some examples.

The reaction sites 6 are illuminated with light from a light source 28. The light passes through an excitation filter 30 before reaching the reaction sites 6. The excitation filter 30 allows light having a specific excitation wavelength to pass through, and blocks other light. Fluorescent molecules at the reaction sites absorb the excitation light and re-emit light having a fluorescent wavelength. The emitted light is reflected from a dichroic beam splitter 32 into a light detector 34. The light detector is behind an emission filter 36 which only allows light having the fluorescent wavelength to pass through, so that any light detected at the light detector 34 may be known to be light at the fluorescent wavelength. In this way, a signal produced by the light detector 34 is indicative of an amount of fluorescent material at the reaction sites 6. Fluorescent molecules may be present at a given reaction site when that reaction site contains bound probe/sample biomolecule pairs. For example, the sample biomolecules may be fluorescent, and are located at the reaction sites when they are bound to those sites via the bound probe molecules as part of a bound probe/sample biomolecule pair. Alternatively, a fluorescent intercalating dye may be bound to the probe/sample biomolecule pairs but not to the probe biomolecules on their own.

Although FIG. 6 illustrates an example in which the detection circuitry is shared between reaction sites, it will be appreciated that in other examples different reaction sites may be provided with different detection circuitry. For example, a light detector 34 may be provided per reaction site or group of reaction sites and there may be separate optical paths from each reaction site or group of reaction sites to a respective light detector.

When probe/sample biomolecule pairs at a given reaction site 6 dissociate, the amount of fluorescence associated with that site may temporarily increase. This is due to reduced quenching of fluorescence as the fluorescent molecules become unbound from the probe molecules at the reaction site 6 but remain in the vicinity of the reaction site 6. This temporary increase in fluorescence reduces as the fluorescent molecules disperse away from the reaction site 6, and a longer term decrease in fluorescence is observed due to a reduction in number of fluorescent molecules bound to the reaction site 6 compared to before the dissociation. The rate of dispersion of fluorescent molecules from the reaction site, and hence the rate at which fluorescence decreases, may be increased using an externally applied electric field.

FIG. 7 is graph showing the amount of fluorescence measured at a site containing bound probe/sample biomolecule pairs during a first heating process. The measurement could be performed using detection circuitry specific to that site or using detection circuitry which detects light across a plurality of sites, with the measured values associated with a particular site due to that site being the site that is being heated. FIG. 7 illustrates the temporary increase and long term decrease in fluorescence at a site following a heating event.

FIG. 8 illustrates the use of fluorescence detection for determining whether each of six sites contain hybridised biomolecules. The six sites each contain a set of probe biomolecules which have been exposed to a set of sample biomolecules under conditions where hybridisation is allowed to take place. A graph is provided for each site indicating the time when heating is applied to that site. It is noted that these graphs are illustrated stacked on top of each other, but have separate axes. For example, at time 0 each heater is provided with 0 current. A fluorescence signal is also illustrated, corresponding to a signal received at the light detector 34 illustrated in FIG. 6. The fluorescence signal is a sum of the fluorescence signals associated with each site, with the detector unable to distinguish between sites so it is not known which sites are responsible for the fluorescence.

The six sites are alternately heated. For example, site 1 is heated first by increasing the power at its heater to raise the temperature at site 1. Any changes in the fluorescence signal during the heating of the first site can be associated with dissociation at the first site, so there is no requirement for the fluorescence signal to be site-specific. The fluorescence signal illustrated in FIG. 8 is simplified and does not illustrate the temporary spikes in fluorescence associated with melting, but does illustrate the longer term decreases in fluorescence when fluorescent molecules are melted off a site (the line can be considered to be an average over a time scale longer than the time scale of the temporary increase).

As illustrated in FIG. 8, the fluorescence signal does not significantly decrease after the heating process at site 1. Therefore, it can be determined that no, or a small number of, fluorescent molecules have been melted from site 1 during its heating event, and therefore that there were no, or a small number of, hybridised probe/sample biomolecule pairs at site 1 after the set of sample biomolecules had been introduced to the sets of probe biomolecules at each site. Therefore, it can be determined that the set of sample biomolecules were not complementary to the set of probe biomolecules at site 1.

However, the fluorescence signal does noticeably decrease after the heating process at site 2. This indicates that site 2 contained a larger number of fluorescent probe/sample biomolecule pairs which have been dissociated by the heating of site 2. Hence, it can be determined that site 2 contained probe biomolecules which were complementary to, and hybridised with, the set of sample biomolecules provided to the sites. Hence, information about which biomolecules were included in the set of sample biomolecules can be gathered, since it is known that at least part of the set of sample biomolecules were complementary to the known probe biomolecule.

This process is continued for each site, and from the changes in the fluorescence signal it can be determined that sites 2, 3, 5, and 6 contained a larger number of hybridised biomolecules whereas sites 1 and 4 contained a smaller number. Therefore, a single measurement value unable to distinguish between sites is able to be used to determine an indication of an amount of hybridisation occurring at specific sites by selectively heating those sites and detecting an amount of dissociation. In addition, the changes detected on dissociation caused by heating have a larger rate of change than those occurring on hybridisation, so less precise circuitry is required to detect dissociation.

Whilst FIG. 8 illustrates the use of decreases in fluorescence to identify the presence of hybridised molecules at sites, it also more generally provides an illustration of the use of a single detection signal, unable to distinguish between sites, for detecting hybridisation at individual sites. This is because each site is heated at different times, so measurements during the period that a particular site is being heated can be associated with that particular site. In a similar way to that illustrated in FIG. 8, the fluorescence signal could be replaced with a signal indicative of charge at the plurality of reaction sites, and peaks in the charge signal occurring during a heating event for a particular site can imply that the particular site contained hybridised biomolecules.

FIG. 9 illustrates the change in fluorescence at a particular reaction site 6 containing hybridised probe/sample biomolecule pairs during four separate heating processes. The fluorescence at a detector 34 is indicated with a solid line and the heater voltage is indicated with a dashed line. It can be seen that in the first heating process, the fluorescence sharply spiked from around 220 a.u. to above 400 a.u. due to the temporary increase in fluorescence associated with a melting event. Similar spikes are not observed for the second to fourth heating processes. Hence, in a similar way to the electrical signals illustrated in FIG. 4, it can be determined that the site initially contained a larger number of probe/sample biomolecule pairs the majority of which were melted during the first heating process. FIG. 10 illustrates the same data as in FIG. 9, with the fluorescence signal isolated during each of the four melting processes, where the spike associated with the first melt can be clearly seen. Hence, either the temporary increase in fluorescence (as shown in FIG. 10) or the longer term decrease in fluorescence (as shown in FIG. 8) can be used to detect dissociation of hybridised pairs, both measurements providing a larger rate of change during dissociation than changes in fluorescence during hybridisation.

It is noted that the longer term decrease in fluorescence is not observed in FIG. 9 because insufficient time was provided between heating processes. If more time were provided, or a stronger external electric field were applied to increase the rate of diffusion of the fluorescent molecules, the fluorescence would be observed to drop below the initial fluorescence value as indicated in FIG. 7.

FIG. 11 illustrates a further example of a reaction site comprising electrical detection circuitry 14. Compared to the example illustrated in FIG. 3, FIG. 11 illustrates two voltage sources 38 providing current to the heater 7 rather than a single voltage source 9. This may help to reduce noise in the electrical detection signal detected by circuitry 14 in cases where the heater 7 is a resistive heater which generates heat due to current passing through an electrical path having a certain amount of electrical resistance. In particular, a single voltage source 9 providing current to the heater 7 may cause charge to be deposited on the reaction site 6. The rate of change of charge caused by the heater may in some examples be larger than the rate of change in charge caused by dissociation of probe/sample biomolecule pairs and may obscure measurements of dissociation. However, if the heater is powered with symmetrical voltage sources, then the net charge coupled to the reaction site 6 by the heater may be reduced. In particular, if the voltage sources each provide an equal and opposite current to parallel resistive heater circuits, then the net rate of charge at the reaction site caused by the heater is reduced since the charge coupled onto the reaction site 6 by a first of the two voltage sources 38 providing current to a first electrical path is cancelled by an equal and opposite amount of charge being coupled off the reaction site by the second of the two voltage sources 38 providing an equal and opposite amount of current to a parallel electrical path. Hence, the rate of change of charge due to the heater may be reduced, while still providing heating corresponding to the combined effect of resistive heating for the two parallel electrical paths, allowing more accurate measurements of the rate of change of charge caused by dissociation.

An alternative method to the use of symmetrical voltage sources 38 is to use a single voltage source 9 as illustrated in FIG. 3, and reduce the rate of change of charge at the reaction site 6 caused by the heater by limiting the amount power provided to the heater (e.g., using a low heater voltage). This may include limiting the rate of change of temperature at the target site. The voltage source for controlling the heater may be selected to be sufficient to cause an increase in the temperature of the heater, but low enough that the rate of change of charge caused by the heater itself at the reaction site 6 is low enough that it does not obscure the rate of change of charge caused by dissociation at the reaction site 6.

FIG. 12 illustrates an example of a reaction site 6 as illustrated in FIG. 11, in which the electrical detection circuitry 14 has been replaced with a first-order sigma-delta analogue to digital converter (ADC) 40. The ADC 40 takes the same input as the detection circuitry 14. However, whilst the detection circuitry 14 outputs a voltage Vₒᵤₜ which is an amplified analogue signal corresponding to the input signal, the ADC 40 outputs a digital signal indicative of the input signal. Conversion of the measured value into digital data can be useful for computational analysis of the signal indicative of hybridisation.

FIG. 13 is a flow diagram illustrating a method for detecting hybridisation by measuring dissociation of hybridised probe/sample biomolecule pairs. At step 1300, a set of probe biomolecules 22 is provided to a reaction site 6 in such a way that the probe biomolecules 22 are bound to the surface of the reaction site 6. Sets of probe biomolecules 22, which may differ from each other, may also be provided to further reaction sites to perform detection at several sites in a single process. The probe biomolecules may be provided to a reaction site 6 by attaching previously produced probe biomolecules to the reaction site 6, or may be synthesised in situ on the reaction site 6.

At step 1302, a set of sample biomolecules 24 is supplied to the reaction site having the set of probe biomolecules 22 provided on it. At step 1304, the set of sample biomolecules is provided with the opportunity to hybridise with the set of probe biomolecules 22 at the site. This means that the conditions at the reaction site 6 may permit hybridisation to take place, the amount of which will be significantly larger if the set of sample biomolecules are complementary to the set of probe biomolecules. An arbitrarily long amount of time may be provided to allow hybridisation to take place (e.g., in excess of the expected time for hybridisation to complete). After step 1304, if the set of sample biomolecules were complementary to the set of probe biomolecules at the reaction site 6, then the site may contain a greater amount of bound probe/sample biomolecule pairs bound to the surface of the reaction site 6. If the biomolecules were not complementary, then a smaller amount of hybridised pairs will be bound to the surface of the reaction site.

At step 1306, the reaction site is heated to a temperature which increases the likelihood of any probe/sample biomolecule pairs at the reaction site 6 dissociating. In particular, the site may be raised above a “melt” temperature for the probe/sample biomolecule pair. This causes dissociation of the probe/sample pairs at the reaction site 6 over a shorter timescale than the initial formation of the probe/sample biomolecule pairs during hybridisation. Physical changes associated with hybridisation may be reversed during dissociation. Hence, dissociation may cause larger rates of change of physical properties than were caused by hybridisation, where the larger rates of change can be easier to measure.

At step 1308, dissociation of probe/sample biomolecule pairs at the reaction site 6 is detected. The dissociation signal is used to generate a hybridisation indication indicating an amount of hybridisation that occurred at the reaction site 6. The dissociation signal is expected to be larger than signals measured during hybridisation due to the higher rate of dissociation than the rate of hybridisation. This allows less precise, and therefore simpler and cheaper, circuitry to be used for hybridisation than in techniques where hybridisation is measured directly. In addition, when there is more than one site dissociation can be caused to happen at different times for different sites and therefore circuitry may be used which is incapable of distinguishing signals from different sites. Again, this allows simpler detection circuitry to be used.

FIGS. 14 and 15 are flow diagrams illustrating in further detail the heating and detection steps 1306 and 1308 of FIG. 13.

At step 1400 of FIG. 14, a temperature increase process is performed at a target reaction site to raise its temperature above a melt temperature of probe/sample biomolecule pairs at the site. The site may be one of a plurality of reaction sites, in which case the temperature increase process may be performed for one reaction site whilst others are maintained at a lower temperature. At step 1402, detection circuitry captures a detection signal indicative of a change in an amount of charge or fluorescence at the target site. At step 1404, a hybridisation indication is generated indicating an amount of hybridisation that previously occurred at the target site (i.e., at step 1304 of FIG. 13) based on the signal detected in step 1402. For example, a significant increase in charge at the reaction site may be indicative of a significant amount of dissociation of probe/sample pairs at the site, which may in turn indicate that hybridisation occurred at the target site. A temporary increase in fluorescence or a longer term decrease in fluorescence may also indicate dissociation, and therefore previous hybridisation. A magnitude of the change in the detected signal may be indicative of an amount of hybridisation such that a larger change may imply that more hybridisation took place than a smaller change. However, in some examples a threshold may be applied to the detected signal such that the hybridisation indication indicates that hybridisation either took place (if the signal exceeds the threshold) or did not (if the signal does not exceed the threshold).

At step 1500 of FIG. 15, the target reaction site is heated in a first temperature increase process. This step may be the same as step 1400 of FIG. 14. At step 1502 a first signal is recorded during the first temperature increase process. At steps 1504 and 1506, the heating and detection processes are repeated without introducing any further sample biomolecules to the target site. Hence, if there were a significant number of probe/sample biomolecule pairs at the target site during the first temperature increase process, there would be expected to be fewer pairs at the target site during the second temperature increase process (having been dissociated during the first temperature increase process). Therefore, the number of dissociating pairs during the second (and further) temperature increase processes would be expected to be less than during the first temperature increase, and hence the dissociation signal would be expected to be smaller. Therefore, if there were a greater number of probe/sample biomolecule pairs at the reaction site then a more significant difference between the two signals (detected at steps 1502 and 1506) would be expected, and measuring a more significant difference between these signals may be indicative of a larger amount of hybridisation. If there were fewer probe/sample biomolecule pairs at the target site prior to step 1500, then the difference between the signals detected at steps 1502 and 1506 would be expected to be the less significant and hence measuring a smaller difference between these signals may indicate that less hybridisation took place.

Hence, at step 1508 a hybridisation indication may be generated based on a difference between the signals recorded at steps 1502 and 1506. Using the difference between these measurements isolates changes in charge or fluorescence due to dissociation from changes in charge or fluorescence caused by other effects which are the same for each temperature increase process, and therefore allows more accurate detection of dissociation with less precise circuitry.

FIG. 16 illustrates a process for detecting hybridisation at a plurality of reaction sites using detection circuitry unable to distinguish between sites.

At step 1600 a set of probe biomolecules are provided at each of the plurality of sites. For example, the probe biomolecules may be attached to, or synthesised on, each of the plurality of sites. When detecting hybridisation by dissociation, at this point a set of sample biomolecules may also be provided to the plurality of reaction sites.

At step 1602, one of the reaction sites of the plurality of reaction sites is selected.

At step 1604, the temperature of the selected reaction site is controlled such that it is different from the temperature of the other reaction sites in the plurality of reaction sites. For example, the selected reaction site may have a higher temperature than the other reaction sites such that dissociation happens primarily at the selected reaction site, or alternatively may have a lower temperature than the other reaction sites such that hybridisation happens primarily at the selected reaction site.

At step 1606 detection circuitry unable to distinguish between sites detects a signal indicative of an amount of hybridisation.

In one example, this signal is a direct measurement of hybridisation. For example, a set of sample biomolecules may be provided to the plurality of reaction sites, but may preferentially hybridise with the selected reaction site as it is at a different temperature than the other reaction sites (a lower temperature than the other sites, for example). Physical changes associated with hybridisation may cause measurable signals such as changes in charge at the reaction site or changes in fluorescence at the reaction site, which can be detected by the detection circuitry. If the set of sample biomolecules is complementary to the set of probe biomolecules at the selected reaction site then a hybridisation signal may be produced which may not be produced if the sets of biomolecules are not complementary. Because the selected reaction site is the site at which hybridisation preferentially occurs due to its different temperature, signals associated with hybridisation can be associated with the selected reaction site even though the detection circuitry is unable to distinguish between sites.

In a second example, the detection signal is an indirect measurement of hybridisation based on detecting dissociation of previously hybridised probe/sample biomolecule pairs. For example, each site may be exposed to sample biomolecules after step 1600, such that sites comprising complementary probe biomolecules which are complementary to the set of sample biomolecules would undergo a greater amount of hybridisation and contain more hybridised pairs than sites comprising probe biomolecules which are not complementary to the set of sample biomolecules. Then, at steps 1604 and 1606 the selected reaction site is heated to a temperature where dissociation is more likely to occur. A signal detected during this process may indicate an amount of dissociation that has occurred at the selected reaction site, and hence an amount of hybridisation that has previously occurred at that reaction site. The signal can be associated with the selected reaction site due to dissociation preferentially occurring at the heated selected reaction site compared to the other reaction sites. Because the number of hybridised biomolecules changes more rapidly during dissociation than during hybridisation, measuring dissociation can produce a larger signal than measuring hybridisation directly and require simpler circuitry. In both examples, the detection circuitry is not required to be able to distinguish different reaction sites in order to associate a hybridisation indication with each site.

At step 1608, the hybridisation indication may be recorded for the selected site. This may be based on information indicating which reaction site was controlled to be a different temperature than the other reaction sites.

At step 1610, it is determined whether there are any further reaction sites and if so the process of steps 1602-1608 is repeated for each additional reaction site in the plurality of reaction sites. If not, then at step 1612 the recorded hybridisation indications may be used to determine which sites have undergone a significant amount of hybridisation to provide an indication of what biomolecules were in the set of sample biomolecules.

FIG. 17 is a graph illustrating the output signals of electrical detection circuitry of two nearby sites in an example thermal control chip comprising a plurality of reaction sites. The reaction sites initially comprise probe/sample biomolecule pairs. One of the sites is heated, whilst the neighbouring site is not heated. FIG. 17 illustrates that the amount of crosstalk, i.e., the signal at the neighbouring site caused by heating of the driven site, is low. In the example of FIG. 17 the crosstalk was measured to be below 6% of the magnitude of the signal of the driven site. This indicates that sites can be accurately isolated with selective heating, with the majority of a measured signal being attributable to the selected site as a result of the low amount of crosstalk.

Although illustrative embodiments of the invention have been described in detail herein with reference to the accompanying drawings, it is to be understood that the invention is not limited to those precise embodiments, and that various changes and modifications can be effected therein by one skilled in the art without departing from the scope of the invention as defined by the appended claims.

1. A method for detecting a hybridisation indication indicative of an amount of hybridisation between a set of probe biomolecules provided at a reaction site and a set of sample biomolecules, the method comprising:
supplying the set of sample biomolecules to the reaction site comprising the set of probe biomolecules;
heating the reaction site to cause dissociation of probe/sample-biomolecule-pairs previously hybridized when the set of sample biomolecules were provided to the reaction site; and
generating the hybridisation indication based on detecting the dissociation of the probe/sample-biomolecule pairs caused by heating the reaction site.

2. The method of claim 1, in which the dissociation of the probe/sample-biomolecule pairs is detected electrically, based on charge released in the dissociation.

3. The method of claim 2, in which temperature control circuitry controls the temperature of the reaction site; and
heating the reaction site comprises suppressing a rate of change of charge at the reaction site caused by the temperature control circuitry.

4. The method of claim 1, in which the detection signal is detected optically, based on a change in fluorescence of the probe biomolecules, the sample biomolecules, or an intercalating dye during the dissociation.

5. The method of claim 1, in which:
the reaction site is one of a plurality of reaction sites of an apparatus comprising temperature control circuitry to independently control temperature for respective reaction sites;
respective reaction sites comprise respective sets of probe biomolecules;
the set of sample biomolecules is provided to each of the plurality of reaction sites; and
a detection process, comprising the heating and generating steps, is alternately performed for each reaction site to provide a plurality of separate hybridisation indications each indicative of an amount of hybridisation between the set of sample biomolecules and the set of probe biomolecules at a respective reaction site.

6. The method of claim 5, in which the reaction sites are provided on a thermal control chip.

7. The method of claim 6, in which the hybridisation indication for a given reaction site is generated, using detection circuitry, based on a detection signal indicative of an amount of dissociation when the given reaction site is heated.

8. The method of claim 7, in which the detection circuitry is shared between the plurality of reaction sites.

9. The method claim 7, in which the detection circuitry is provided within or outside the boundary of the thermal control chip.

10. The method of claim 7, in which the detection signal is incapable of resolving which of the plurality of reaction sites is the given reaction site at which the probe/sample-biomolecule pairs dissociated.

11. The method of claim 10, in which the hybridisation indication for the given reaction site is generated based on the detection signal and information indicative of which reaction site was heated when the detection signal was detected by the detection circuitry.

12. The method of claim 1, in which the set of probe biomolecules is synthesized in situ on the reaction site prior to supplying the set of sample biomolecules.

13. The method of claim 12, in which the reaction site is one of a plurality of reaction sites of an apparatus comprising temperature control circuitry to independently control temperature for respective reaction sites; and
synthesis of a respective set of probe biomolecules at respective reaction sites is thermally controlled by the temperature control circuitry independently controlling temperatures at each reaction site.

14. The method of claim 1, wherein the probe biomolecules comprise at least one of:
single-stranded DNA, RNA or another nucleic acid;
synthesis initiators for DNA, RNA or another nucleic acid; and
peptides, DNA peptide conjugates, or peptide hybrids with another nucleic acid.

15. The method of claim 1, wherein the sample biomolecules comprise at least one of:
single-stranded DNA, RNA or another nucleic acid; and
peptides, DNA peptide conjugates, or peptide hybrids with another nucleic acid.

16. The method of claim 1, in which at least one of the set of probe biomolecules and the set of sample biomolecules comprises double-stranded or single-stranded DNA, RNA or another nucleic acid.

17. The method of claim 1, wherein a process of heating the reaction site is performed two or more times, and the hybridisation indication is generated based on a comparison between dissociation detected during a first heating process and dissociation detected during a subsequent heating process.

18. Use of a thermal control chip, comprising a plurality of reaction sites and temperature control circuitry to independently control temperature for respective reaction sites, for increasing signal strength in detection of hybridisation between a set of sample biomolecules and respective sets of probe biomolecules provided at the respective reaction sites.

19. A method for detecting hybridisation between a set of sample biomolecules and respective sets of probe biomolecules provided at respective reaction sites of an apparatus comprising temperature control circuitry to independently control temperature for respective reaction sites; the method comprising:
for each reaction site alternately selected as a selected reaction site, alternately performing a detection process comprising:
controlling the temperature at the respective reaction sites to set the selected reaction site to a different temperature to other reaction sites;
detecting a detection signal indicative of an amount of hybridisation between the set of probe biomolecules and the sample biomolecules, wherein the detection signal is incapable of resolving which particular site is the selected reaction site at which the hybridisation or dissociation occurred; and
based on the detection signal, recording a hybridisation indication associated with the selected reaction site.

20. The method of claim 19, comprising determining, based on the hybridisation indications recorded for each reaction site, which of the reaction sites have undergone hybridisation between the set of sample biomolecules and a corresponding set of probe biomolecules.

21-26. (canceled)
